(19) United States
(12) Patent Application Publication
(10) Pub. No.: US 2024/0139538 A1
(43) Pub. Date: May 2, 2024

(54) LIGHT THERAPY SYSTEM

(71) Applicant: Mark Sawyer, Lake Mary, FL (US)

(72) Inventor: Mark Sawyer, Lake Mary, FL (US)

(21) Appl. No.: 18/544,172

(22) Filed: Dec. 18, 2023

Related U.S. Application Data
(63) Continuation-in-part of application No. 17/961,258, filed on Oct. 6, 2022.
(60) Provisional application No. 63/354,958, filed on Jun. 23, 2022.

(51) Int. Cl. A61N 5/06

(52) U.S. Cl. CPC A61N 5/06; A61N 2005/0626; A61N 2005/0653; A61N 2005/0663

(57) ABSTRACT

A light therapy system includes a remote-control device and at least one light therapy device. The light therapy devices operate in a standalone mode using the onboard interface device and a cumulative mode where operating instructions are received from the remote-control device. The operating instructions are distinct and individual for each light therapy device, to permit identical or different operation between devices. The light therapy devices do not communicate directly with each other and are operated wirelessly by the remote-control device such that a failure in one light therapy device does not affect the operation of another light therapy device. A hanging assembly suspends a device in a level orientation, and a connection panel secures two devices together such that the light emissions from both devices are oriented parallel to each other and do not overlap.

### CROSS-REFERENCE TO RELATED APPLICATIONS

This application is a continuation in part to and claims the benefit of U.S. application Ser. No. 17/961,258 filed on Jan. 10, 2023, the contents of which are incorporated herein by reference.

### TECHNICAL FIELD

The present invention relates generally to therapy devices, and more particularly to a light therapy system having one or more devices that are securely arranged to precisely deliver light therapy to a user.

### BACKGROUND

The statements in this section merely provide background information related to the present disclosure and may not constitute prior art.

Lighting units for use in agricultural settings have been used for decades to generate photons for use by plants for photosynthesis. More recently, scientific research has shown that exposing humans to certain types of light can have a meaningful effect on a person's overall health. Depending on the intensity and wavelength of the light, these effects can include reducing stress, eliminating headaches, promoting hair growth, increasing collagen production, and even fat reduction.

For these reasons, there are several known types of light therapy devices currently on the market. Such devices typically comprise a panel having an array of individual lights that are controlled by an onboard controller. A switch located directly on the panel is physically activated by a user to transition the device between an ON and OFF operating state.

Traditionally, such devices are manufactured to include a height of about 18 inches and a width of about 8 inches so as to be suitable for providing light therapy to a single area of a user's body such as the head, chest, or back, for example. In this regard, it is important that a user be able to accurately position the device to ensure the desired portion of their body receives the treatment.

As shown at background FIG. 1A, traditional light therapy devices typically include a mounting kit having a pair of posts 1 that are screwed into the top surface of the device 2 and are connected to a pair of elongated cords 3 that extend diagonally to engage a carabiner 4 which itself is connected to a rope 5 and pulley system 6. Although useful for simply hanging a device, many users complain that it is difficult to securely align and position the device with any degree of accuracy. To this end, it is not uncommon for one of the posts 1 to be screwed into the device at a different height relative to the other, or for the cords 3 to slip or move along the carabiner 4. As shown by arrow a, when this occurs, the device 2 will not be level, and the treatment area of the user will be impacted.

In addition to the above, it is not uncommon to link two or more devices together to allow a user to treat multiple areas of their body at once. In order to function optimally, each of the connected devices must operate at the same output levels and must be precisely oriented such that the light output of one device does not overlap the light output of another device.

As shown at background FIG. 1B, the traditional way to link two devices together is by positioning the posts 1 of the lower device 2′ into a pair of keyhole openings on the bottom wall of the upper device 2 such that the second device 2′ hangs loosely from the bottom of the top device 2. Unfortunately, this loose hanging arrangement does not afford any resistance to the pivotal movement of the two devices relative to each other, and often results in the two devices being oriented differently. As a result, the light output L from the top device will often overlap with the light output L′ from the bottom device, thus reducing or eliminating the advantages of using multiple devices at once.

Accordingly, it would be beneficial to provide a light therapy system that can be precisely hung and oriented so as to overcome the above noted issues for both a single and multi-device operation.

### SUMMARY OF THE INVENTION

The present invention is directed to a light therapy system. One embodiment of the present invention can include a remote-control device and at least one light therapy device. Each of the light therapy devices can include a main body, a plurality of optical emitters, a system controller, and an onboard user interface device.

In one embodiment, the plurality of light therapy devices can operate in a standalone mode wherein operating instructions are received from the onboard interface device or the remote-control device and implemented by the onboard controller. In one embodiment, the plurality of light therapy devices can also operate in a cumulative mode where operating instructions are received from the remote-control device. The operating instructions can be distinct and individual for each light therapy device, so as to allow each device to operate in the same manner or in a different manner than the other light therapy devices that are connected to the remote-control.

When operating in the cumulative mode, the plurality of light therapy devices does not communicate directly with each other, and can be operated individually and wirelessly by the remote-control device such that a failure in one light therapy device does not affect the operation of another light therapy device.

One embodiment of the system also includes a hanging assembly having a panel bracket that is secured to the back side of a light therapy device. The assembly can function to ensure the device is suspended in a completely level orientation for precisely aiming light emissions from the device. The system can also include multi-device connection panel that aligns and secures two light therapy devices together vertically such that the light emissions from both devices are oriented parallel to each other and do not overlap.

This summary is provided merely to introduce certain concepts and not to identify key or essential features of the claimed subject matter.

### BRIEF DESCRIPTION OF THE DRAWINGS

Presently preferred embodiments are shown in the drawings. It should be appreciated, however, that the invention is not limited to the precise arrangements and instrumentalities shown.

FIG. 1A is a front view of a hanging system for light devices in accordance with the background art.

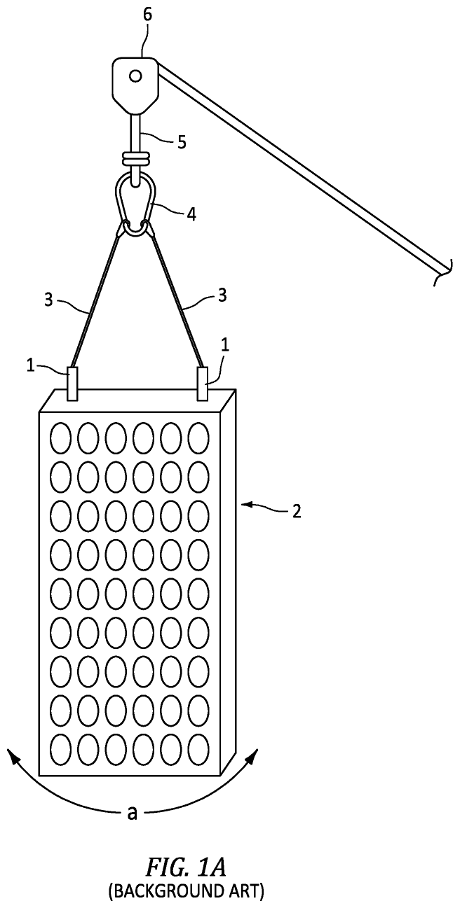

FIG. 1B is a side view of the connection mechanisms for connecting two light devices together in accordance with the background art.

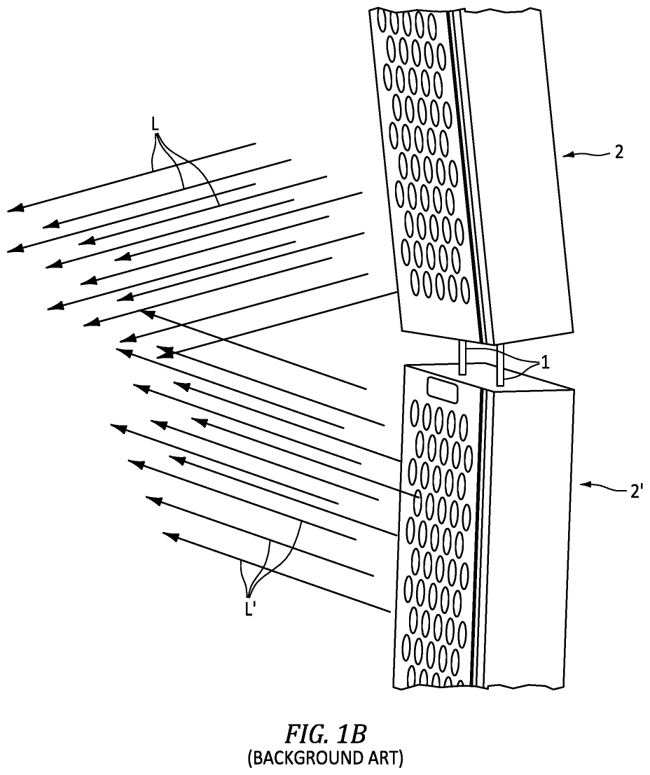

FIG. 2A is a perspective view of a light therapy system in accordance with one embodiment of the invention.

FIG. 2B is a perspective view of a light therapy device of the light therapy system, in accordance with one embodiment of the invention.

FIG. 3 is a simplified block diagram of the system controller of the light therapy device, in accordance with one embodiment of the invention.

FIG. 4 is another perspective view of the light therapy system, in accordance with one embodiment of the invention.

FIG. 5A is a perspective view of a hanging assembly of the light therapy system in operation, in accordance with one embodiment of the invention.

FIG. 5B is a front view of a hanging assembly of the light therapy system in operation, in accordance with one embodiment of the invention.

FIG. 6A is a perspective view of a multi-device connection panel of the light therapy system in operation, in accordance with one embodiment of the invention.

FIG. 6B is a back view of a multi-device connection panel of the light therapy system in operation, in accordance with one embodiment of the invention.

FIG. 6C is a perspective view of the light therapy system in operation, in accordance with one embodiment of the invention.

### DETAILED DESCRIPTION OF THE INVENTION

While the specification concludes with claims defining the features of the invention that are regarded as novel, it is believed that the invention will be better understood from a consideration of the description in conjunction with the drawings. As required, detailed embodiments of the present invention are disclosed herein; however, it is to be understood that the disclosed embodiments are merely exemplary of the invention which can be embodied in various forms. Therefore, specific structural and functional details disclosed herein are not to be interpreted as limiting, but merely as a basis for the claims and as a representative basis for teaching one skilled in the art to variously employ the inventive arrangements in virtually any appropriately detailed structure. Further, the terms and phrases used herein are not intended to be limiting but rather to provide an understandable description of the invention.

### Definitions

As described herein, a “unit” means a series of identified physical components which are linked together and/or function together to perform a specified function.

As described throughout this document, the term “about” “approximately” “substantially” and “generally” shall be used interchangeably to describe a feature, shape, or measurement of a component within a tolerance such as, for example, manufacturing tolerances, measurement tolerances or the like.

As described throughout this document, the term “complementary” shall be used to describe a shape, size and/or location of a component that is identical to, or substantially identical to the shape, size and/or location of another identified component within a tolerance such as, for example, manufacturing tolerances, measurement tolerances or the like.

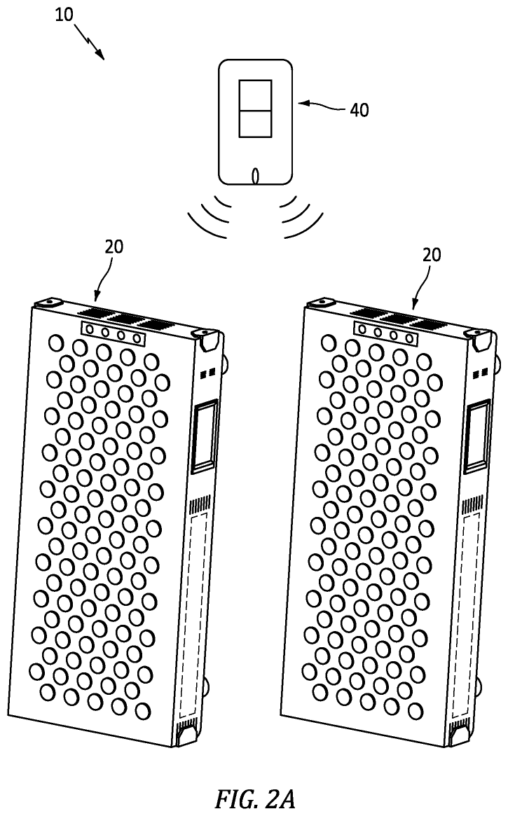

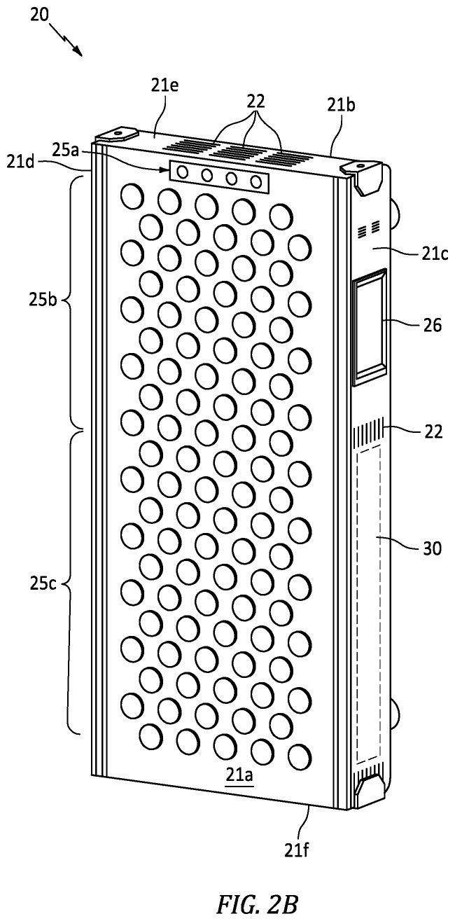

FIGS. 2A-6C illustrate one embodiment of a light therapy system 10 that are useful for understanding the inventive concepts disclosed herein. In each of the drawings, identical reference numerals are used for like elements of the invention or elements of like function. For the sake of clarity, only those reference numerals are shown in the individual figures which are necessary for the description of the respective figure. For purposes of this description, the terms “upper,” “bottom,” “right,” “left,” “front,” “vertical,” “horizontal,” and derivatives thereof shall relate to the invention as oriented in FIG. 2B.

As shown and described herein, the light therapy system 10 can include one or more individual light therapy devices 20, a remote-control device 40, a hanging assembly 50, and a multi-device connection member 60.

As shown best at FIG. 2B, each light therapy device 20 can include a main body having a front wall 21a, a back wall 21b, a pair of side walls 21c and 21d, a top wall 21e and a bottom wall 21f that define an interior space. In the preferred embodiment, the main body will be constructed from stamped steel panels and will include a generally rectangular shape having a plurality of air vents 22 along each of the top, bottom, back and side walls.

Of course, other embodiments are contemplated wherein the main body includes any number of different shapes and sizes. Moreover, the main body may be formed from any number of other materials that are, for example, relatively strong and stiff for their weight. Several nonlimiting examples include but are not limited to various metals or metal alloys (e.g., aluminum, titanium, or alloys thereof), plastic/polymers (e.g., high-density polyethylene (HDPE), rigid polyvinyl chloride (PVC), or polyethylene terephthalate (PET)), and/or various composite materials (e.g., carbon fibers in a polymer matrix, fiberglass, etc.).

In one embodiment, a plurality of optical emitters can be positioned along or within the main body so as to direct optical emissions outward from the front surface 21a in a specified direction. In the preferred embodiment, the optical emissions will be oriented directly perpendicular to the major axis of the main body, however other orientations are contemplated. Each of the optical emitters can be communicatively linked to the below described controller 30 in order to receive operating instructions and/or power therefrom. In the preferred embodiment, each of the optical emitters can comprise light emitting diodes (LED's); however, any number of other types of devices capable of producing optical emissions are also contemplated.

In one embodiment, the plurality of optical emitters can be divided into different groups or arrays such as arrays 25a, 25b and 25c, for example. To this end, each array can produce an optical output/output emission that is the same as another array or that is different from another array. For example, in one nonlimiting embodiment, array 25a can function to produce visible red light at any desirable wavelength suitable for visible red-light therapy, whereas array 25b can function to produce infrared light at any desirable wavelength suitable for infrared therapy.

In the same nonlimiting exemplary embodiment, array 25c can produce visible light in any number of different colors and intensities so as to provide a visual indication to a device user of the operating mode at which the device is operating. The presence of array 25c on the front wall of the device is specifically advantageous, as it allows a user to constantly monitor the operating status of the device without having to look away or move to the side of the device to see the control panel.

Although described above with regard to a specific number of arrays operating at specific outputs, this is for illustrative purposes only. To this end, each device 20 can include any number of different arrays each having any number of different optical emitters capable of producing light in any color and in any of the visible, near-infrared, infrared, far-infrared and/or other spectrums. Moreover, some of the optical emitters can include a different construction than other emitters. Several nonlimiting examples of other types of optical emitters which may be used herein include heat bulbs, incandescent bulbs, LCD, and/or OLED elements, among others, for example. Such a feature allows a single device to be capable of performing light therapy at multiple different wavelengths and emission parameters at the same time so as to treat different ailments simultaneously.

In one embodiment, a user interface 26 can be communicatively linked to the system controller 30 and can be positioned along one of the walls of the main body. In the preferred embodiment, the user interface device can include a Graphic User Interface (GUI) screen for providing two-way communication with a user. To this end, the interface can allow the user to instruct the system to perform functions such as transitioning between an on and off operating state, selectively activating individual optical emitters, selectively activating specific arrays of optical emitters, and for selecting the type and durational output of the same.

In one embodiment, the user interface can include functionality for allowing the user to designate a plurality of specific optical emitters 25 in order to create one or more user-defined arrays. For example, in one nonlimiting embodiment, the user can create a first array comprising the top 4 rows of emitters, and a second array comprising the bottom 4 rows of emitters. Each of the user-defined arrays being capable of operating in the same manner described above with regard to arrays 25a-25c wherein each array can produce output emissions that are the same as another user-defined array or that are different from another user-defined array.

Of course, any number of other user-defined arrays can be created by the user via the user interface device. Such a feature advantageously provides a fully user customizable light therapy device which can be tailored to the specific and different requirements of a user each time they use the device. Additionally, the user interface device is not limited to the use of a touch screen system, as any number of other types of devices such as non-touch screen displays, buttons, switches and/or other input mechanisms are also contemplated.

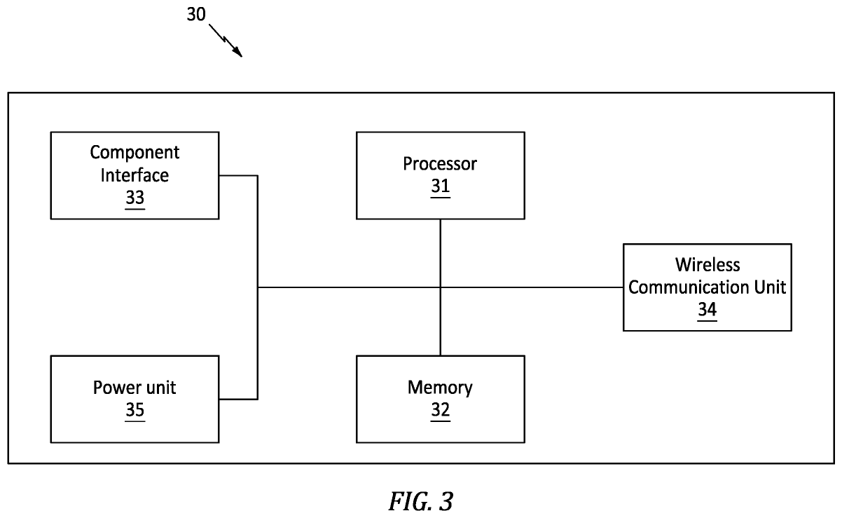

FIG. 3 is a simplistic block diagram illustrating one embodiment of the controller 30 that includes a processing unit 31 that is conventionally connected to an internal memory 32, a component interface unit 33, a wireless communication unit 34, and/or a power unit 35.

Although illustrated as separate elements, those of skill in the art will recognize that one or more system components may comprise or include one or more printed circuit boards (PCB) containing any number of integrated circuit or circuits for completing the activities described herein. The CPU may be one or more integrated circuits having firmware for causing the circuitry to complete the activities described herein. Of course, any number of other analog and/or digital components capable of performing the described functionality can be provided in place of, or in conjunction with the described elements.

The processing unit 31 can include one or more central processing units (CPU) or any other type of device, or multiple devices, capable of manipulating or processing information such as program code stored in the memory 32 in order to allow the device to perform the functionality described herein.

Memory 32 can act to store operating instructions in the form of program code for the processing unit 31 to execute. Although illustrated in FIG. 3 as a single component, memory 32 can include one or more physical memory devices such as, for example, local memory and/or one or more bulk storage devices. As used herein, local memory can refer to random access memory or other non-persistent memory device(s) generally used during actual execution of program code, whereas a bulk storage device can be implemented as a persistent data storage device such as a hard drive, for example. The bulk storage device can contain any number of different programs that permit the processor to perform the functionality described herein and can also receive and store the exercise information for each user.

The component interface unit 33 can function to provide a communicative link between the processing unit 31, the optical emitters 25 and the user interface device 26. In this regard, the component interface unit can include any number of different components such as one or more PIC microcontrollers, standard bus, internal bus, connection cables, wireless receiver and/or associated hardware such as USB cables and connectors, and other such hardware capable of linking the various components. Of course, any other means for providing two-way communication between the system components can also be utilized herein.

The communication unit 34 can include any number of components capable of sending and/or receiving electronic signals with another device, either directly or over a network. In the preferred embodiment, the communication unit 34 can include a Wi-Fi transceiver for communicating directly with the remote-control device 40. In another embodiment, the communication unit 34 can include a Wi-Fi or Bluetooth transceiver which can communicate with a user device such as a tablet or smartphone, for example, that is running a mobile application that can be downloaded onto the user device and installed as an application.

Of course, any number of other transmission and reception mechanisms and protocols can also be utilized herein, several nonlimiting examples include cellular transceivers, Near-Field-Communication (NFC) devices, radio, infrared (IR), RFID, and/or network adapter(s) functioning to communicate over a WAN, LAN, or the internet via an internet service provider, among others, for example.

The power unit 35 can include any number of different components capable of providing the necessary power requirements to each element of the device. In the preferred embodiment, the power source can include or comprise an A/C electrical power transformer and cord for allowing the device to be plugged into a standard electrical outlet. Of course, other embodiments are contemplated wherein the power unit includes onboard batteries so as to operate without the need to connect to a power outlet.

In operation, each of the light therapy devices 20 can operate independently via the user interface device so as to function in a standalone mode. Additionally, each of the light therapy devices can operate in a cumulative mode wherein each device is controlled simultaneously by the below described remote-control device.

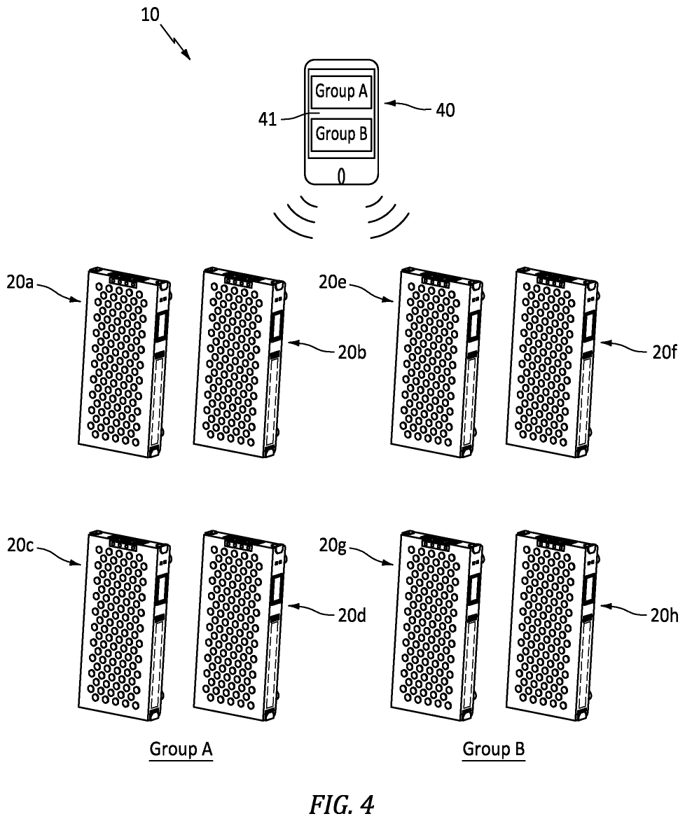

As shown best at FIG. 4, the remote-control device 40 can function to selectively control the operation of any number of individual light therapy devices both individually and as a group. To this end, the remote-control device can include onboard circuitry such as a processor, memory, component interface, wireless interface, and power source substantially identical to that described above with regard to the system controller of the light therapy device.

In one embodiment, the remote-control device can include a small portable housing having a user interface 41 such as individual buttons or a Graphic User Interface (GUI) screen for receiving user instructions and for wirelessly sending operating instructions to one or more of the light therapy devices. Several nonlimiting examples of operating instructions include, but are not limited to a device pairing instruction, device operating mode instructions, device array selection instructions, and/or device array creation instructions, among others for example.

In one embodiment, the system 10 can be configured so that the remote-control device operates in an “on demand” mode. In this configuration, each light therapy device will start, stop, or change a respective operation only upon receipt of an instruction from the remote-control device 40, and will otherwise continue to operate in its previously instructed state until another instruction is received.

In another embodiment, the system can be configured so that the remote-control device operates in a continuous or semi-continuous mode. In this configuration, each light therapy device will automatically transition to an OFF operating state if a signal from the remote-control device is not received within a predetermined timeframe such as every 30 seconds, for example. Such a feature allows the remote-control device to function with other types of light therapy devices that do not have an onboard memory and therefore must receive continuous instructions to continue operating in a desired manner.

By automatically terminating the operation of each light therapy device in the absence of a contrary instruction from the remote-control device, the system operating in the continuous or semi-continuous mode advantageously provides a built-in safety mechanism for ensuring users are not exposed to the therapy device for longer than the recommended timeline.

When used with a plurality of individual therapy devices 20 in the cumulative mode, the remote-control 40 can function to send individualized operating instructions to each device at the same time, in order to allow each device 20 to operate in unison with the other system light therapy devices. Moreover, because each of the light therapy devices are communicatively isolated from each other—e.g., no individual light therapy device is connected to, controls or otherwise communicates with another light therapy device—the system advantageously ensures that a failure by any one light therapy device will have no effect on any of the other light therapy devices in the system.

Additionally, because the light therapy devices are not physically connected together, it is possible to position each device anywhere within a room or other location without being constrained by the positioning of communication cables, as the distances are limited only by the range of the wireless transceiver of the remote-control unit.

In one embodiment, the remote-control can include functionality for allowing a remote-control user to individually select and group different light therapy devices together to form any number of device groupings (e.g., individualized operating instructions). For example, if the remote-control is operating eight light therapy devices 20a, 20b, 20c, 20d, 20e, 20f, 20g and 20h, the user can group several of those into a first group “Group A”, and the remaining devices into a second group “Group B”. In this regard, the remote-control can send different instructions to the different groups based on any number of user criteria.

The ability to send these individualized operating instructions to multiple devices via a single remote is particularly useful in a commercial setting such as a therapist office, for example, wherein multiple light therapy devices are used in different treatment rooms for treating different patients simultaneously. Such a feature allowing a single remote-control device to direct the operation of each group of light therapy devices in each treatment room so as to start, stop or change the operating mode without interfering with the operation of the other groups located in a different therapy room.

Accordingly, the above described system functions in a novel manner by providing a system that allows a user to: 1) configure a plurality of light therapy devices to each operate in a user customized manner via predetermined or user-customized arrays of optical emitters, 2) position each of the light therapy devices in any desirable location, 3) individually control each device via a single remote-control device such that a failure in one device does not affect the operation of another device, and 4) separate the plurality of user-customized devices into user customized groups.

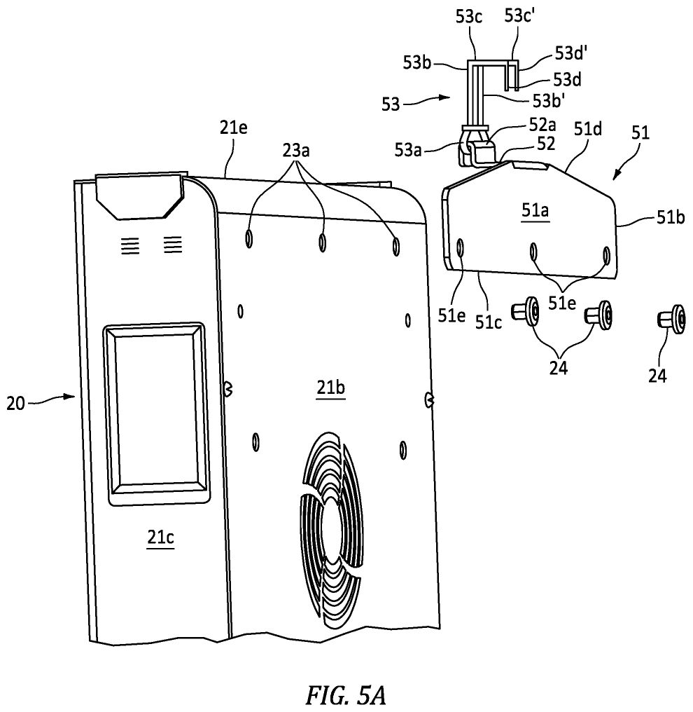

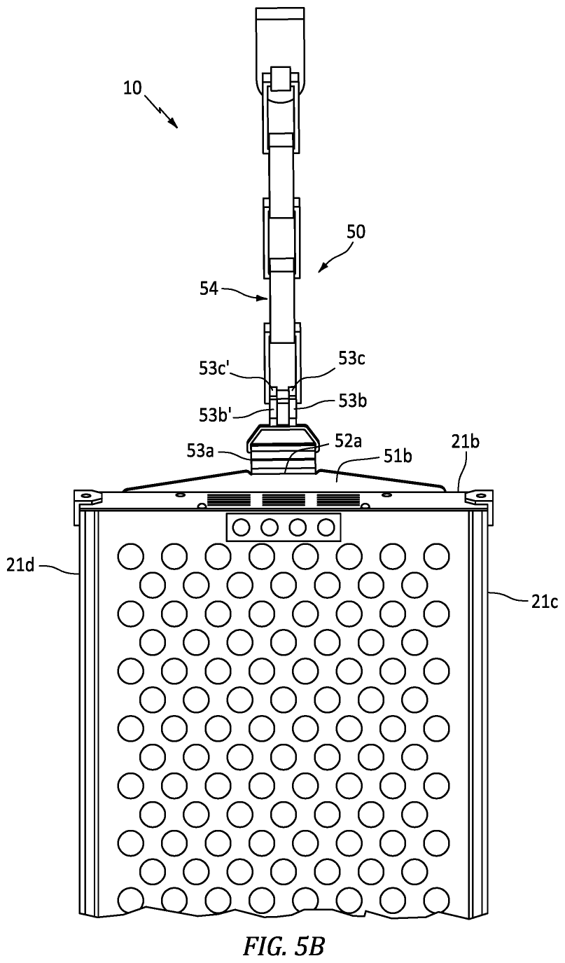

FIGS. 5A and 5B illustrate one embodiment of a hanging assembly 50 for securing a light therapy device 20 onto a door or other structure in a secure and level manner. As shown, the assembly can include a hanging bracket 51, a hook 53, a chain 54 and a door mount 55.

The hanging bracket 51 includes a back surface 51a, a front surface 51b, a bottom wall 51c and an angled top wall 51d. Three openings 51e are provided adjacent to the bottom wall of the bracket and are positioned at complementary locations to threaded apertures 23a located along the upper end of the back wall 21b of the light therapy device 20. The bracket is secured to the device via mounting screws 24 that pass through the openings to engage the threaded apertures.

In one embodiment, the bracket includes an elongated protrusion 52 that extends perpendicularly from the front surface 51a along the center of the top wall 51d. As shown, an elongated receptacle 52a is positioned along the distal end of the protrusion. The receptacle includes a flat channel for receiving the flat bottom rod 53a of a hook 53. In the preferred embodiment, the receptacle 52a and bottom rod 53a will comprise flat straight members having a width of at least three inches. Such features are important to ensure the device remains completely vertical and will not swing or move angularly (e.g., left or right with a combined upward or downward motion) when suspended from the assembly components.

The top portion of the hook 53 can include two engagement members having a first vertical arm 53b and 53b′, a horizontal arm 53c and 53c′ and a second vertical arm 53d and 53d′, respectively. Each arm segment of each engagement member preferably being arranged at 90 degree bends relative to an adjacent arm, and each of the engagement members being substantially identical to each other.

In one embodiment, an elongated square link chain 54 is provided to connect the hook to the door mount. As is known, square link chains comprise a series of square or rectangular shaped links. In operation, the horizontal arms 53b and 53b′ are positioned through one link of the chain at a user-desired height. Importantly, because each link in the chain is square or rectangular, the horizontal walls of each link are flat and arranged level. As such, when the two-level horizontal arms are secured therein, the device 20 is suspended level.

The door mount 55 is designed to engage the top edge of a door or other structure to suspend one or more light therapy devices therefrom. In the preferred embodiment, the door mount includes an inverted and generally U-shaped channel 55a, a vertical body section 55b and a square protrusion 55c extending outward therefrom. The square protrusion having a flat upper surface that engages the flat upper horizontal wall of the uppermost link of the chain 54.

In this regard, when the mount 55 is secured onto a door 7, the horizontal walls of each chain link are arranged level (e.g., both ends are at the same height), the horizontal arms of the hooks are level, and the top wall 21e of the device 20 is level.

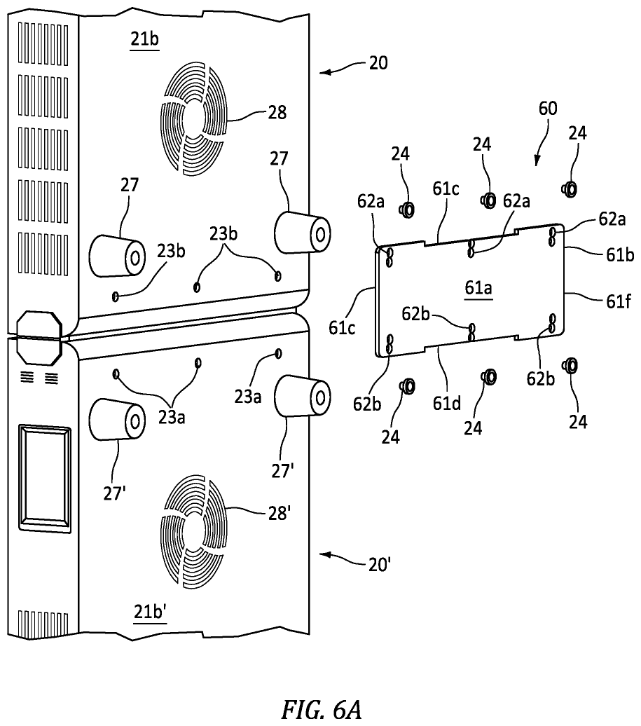

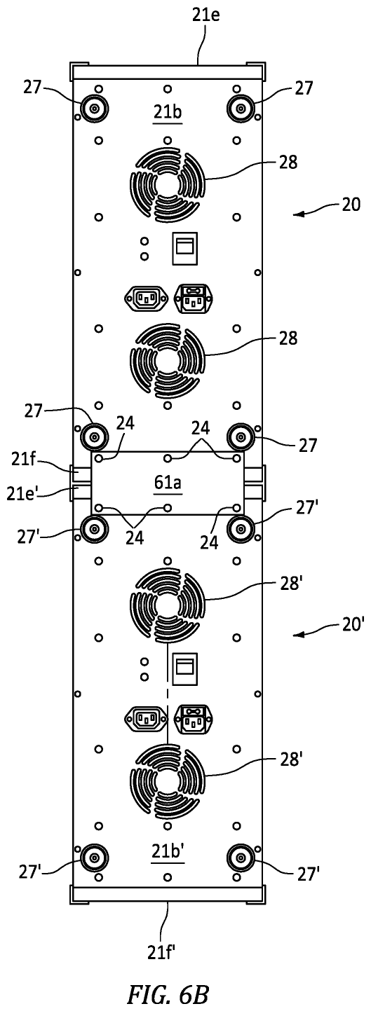

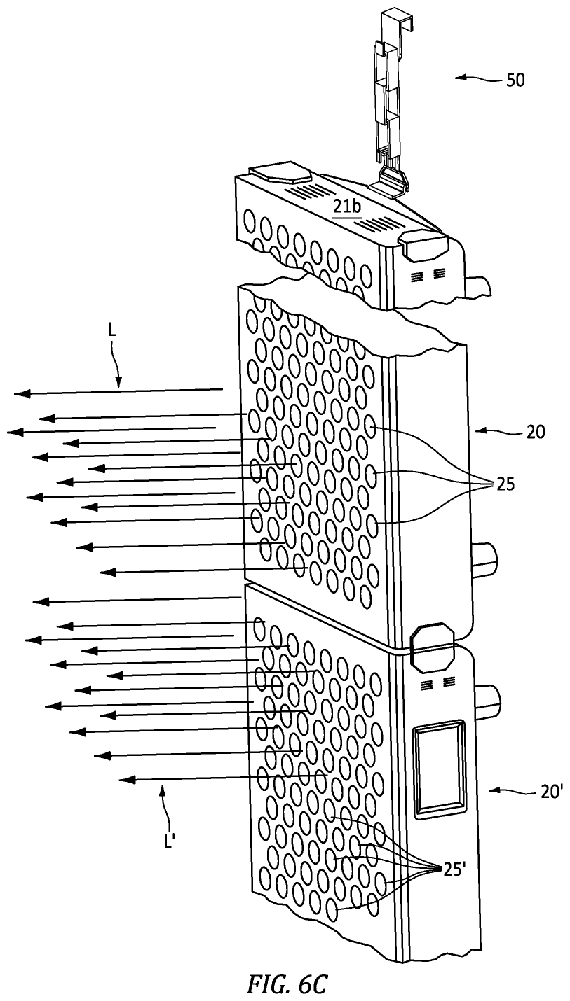

FIGS. 6A-6C illustrate one embodiment of a multi-device connection member 60 for securing two light therapy devices 20 and 20′ together such that the optical emissions of the respective devices will not overlap. As used herein, light therapy devices 20 and 20′ being identical. As shown, the connection member can include a flat, generally rectangular-shaped plate having a back surface 61a, a front surface 61b, a top wall 61c, a bottom wall 61d and a pair of side walls 61e and 61f.

In one embodiment, a plurality of upper openings 62a are positioned along the plate adjacent to the top wall, and a plurality of lower openings 62b are positioned along the plate adjacent to the bottom wall. Upper openings 62a are positioned complementary to a plurality of lower threaded apertures 23b located along the bottom end of the back wall 21b of an upper light therapy device 20, and lower openings 62b are positioned at locations complementary to upper threaded apertures 23a′ located along the top end of the back wall 21b′ of a lower light therapy device 20′. The plate is secured to both devices 20 and 20′ via additional mounting screws 24 that pass through the openings to engage the threaded apertures.

When so positioned, the entire top wall 21e′ of the lower device 20′ will be in direct contact with the entire bottom wall 21f of the upper device 20. Such a feature provides a completely stable configuration that aligns the front walls 21a and 21a′ in a completely linear orientation (e.g., the angles of both devices are exactly the same). Moreover, when the devices are suspended via the hanging assembly 50, the entire system will be positioned and remain completely level. As such, when the optical emitters 25 and 25′ output light, the light emissions L and L′ from both devices remain parallel and do not overlap.

The plate 60 is preferably constructed from a rigid material such as aluminum or steel, for example, so as to resist and prevent tortional forces and/or bending in any direction.

In order to function with multi-system components, it is preferred that the plate include a height (e.g., distance between top wall 61c and bottom wall 61d) of 3.5 inches so as to enable the stands 27 and 27′ of each device to remain affixed to the main body. Such a feature advantageously provides a fixed separation distance between the back surfaces 21b and 21b′ of the devices and the door or wall to which the devices are secured, in order to permit airflow into the fan assemblies 28 and 28′. Likewise, the plate will include a width (e.g., distance between the side walls 61c-61d) that is less than the width of each device (e.g., walls 21c-21d).

As to a further description of the manner and use of the present invention, the same should be apparent from the above description. Accordingly, no further discussion relating to the manner of usage and operation will be provided.

As described herein, one or more elements of each of the light therapy devices can be secured together utilizing any number of known attachment means such as, for example, screws, glue, compression fittings and welds, among others. Moreover, although the above embodiments have been described as including separate individual elements, the inventive concepts disclosed herein are not so limiting. To this end, one of skill in the art will recognize that one or more individually identified elements may be formed together as one or more continuous elements, either through manufacturing processes, such as welding, casting, or molding, or through the use of a singular piece of material milled or machined with the aforementioned components forming identifiable sections thereof.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Likewise, the term “consisting” shall be used to describe only those components identified. In each instance where a device comprises certain elements, it will inherently consist of each of those identified elements as well.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A light therapy system, comprising:
a remote-control device;
a first light therapy device having a main body, a plurality of optical emitters, a first system controller and a first user interface device;
a second light therapy device that is removably connected to the first light therapy device, said second light therapy device having a main body, a plurality of optical emitters, a second system controller and a second user interface device;
a hanging bracket that is removably connected to the first light therapy device;
a hook that is removably connected to the hanging bracket;
a chain that is removably connected to the hook; and
a door mount that is removably connected to the hook.

2. The system of claim 1, wherein the first light therapy device further comprises:
a bottom plurality of threaded openings that are positioned along a back surface adjacent to a bottom wall; and an upper plurality of threaded openings that are positioned along the back surface adjacent to a top wall.

3. The system of claim 2, wherein the hanging bracket includes a plurality of holes.

4. The system of claim 3, wherein the plurality of holes on the hanging bracket include a complementary shape and location as the upper plurality of threaded openings on the first light therapy device.

5. The system of claim 4, wherein the hanging bracket is secured to the back surface of the first light therapy devices via screws that extend through the plurality of holes and engage the plurality of threaded openings.

6. The system of claim 2, further comprising:
a connection plate having a flat metallic body, an upper plurality of holes, and a lower plurality of holes.

7. The system of claim 6, wherein the upper plurality of holes on the connection plate include a complementary shape and location as the lower plurality of threaded openings on the first light therapy device.

8. The system of claim 7, wherein the lower plurality of holes on the connection plate include a complementary shape and location as the upper plurality of threaded openings on the second light therapy device.

9. The system of claim 8, wherein the connection plate is secured to the back surface of the first light therapy device via a first plurality of screws that extend through the upper plurality of holes and engage the lower plurality of threaded openings; and
is secured to the back surface of the second light therapy device via a second plurality of screws that extend through the lower plurality of holes and engage the upper plurality of threaded openings.

10. The system of claim 9, wherein the connection plate functions to secure the first light therapy device to the second light therapy device and to prevent independent movement of the first light therapy device relative to the second light therapy device.

11. The system of claim 1, further comprising:
a remote control device that includes functionality for communicating wirelessly with each of the first light therapy device and the second light therapy device.

12. The system of claim 11, wherein the remote-control device is configured to send individualized operating instructions to each of the first light therapy device and the second light therapy device.

13. The system of claim 11, wherein when operating in a cumulative mode, an operating instruction received from the remote-control device will override an input by the first user interface device affecting the operation of the first light therapy device.

14. The system of claim 13, wherein when operating in a cumulative mode, an operating instruction received from the remote-control device will override an input by the second user interface device affecting the operation of the second light therapy device.
